(19) United States
(12) Patent Application Publication
(10) Pub. No.: US 2026/0041490 A1
(43) Pub. Date: Feb. 12, 2026

(54) SYSTEM AND METHOD FOR POSITIONING OF AUGMENT IN GLENOID SURGERY

(71) Applicant: ORTHOSOFT ULC, Montreal (CA)

(72) Inventors: Karine DUPUIS, Montreal (CA); Ian BASTA, Montreal (CA); Julie DESLONGCHAMPS, Montreal (CA)

(21) Appl. No.: 19/361,841

(22) Filed: Oct. 17, 2025

Related U.S. Application Data
(63) Continuation-in-part of application No. 17/088,713, filed on Nov. 4, 2020, now abandoned.
(60) Provisional application No. 62/930,289, filed on Nov. 4, 2019.

(51) Int. Cl. A61B 34/10; A61B 17/00; A61B 17/56; A61B 34/00

(52) U.S. Cl. CPC A61B 34/10; A61B 17/56; A61B 34/25; A61B 2017/00526; A61B 2017/564; A61B 2034/102; A61B 2034/105; A61B 2034/107

(57) ABSTRACT

Patient-specific instrumentation for reverse shoulder surgery includes a jig having a contact surface including a patient-specific surface portion negatively shaped as a function of a glenoid surface and configured to be applied against the glenoid surface in unique complementary engagement. A first throughbore opens into the contact surface, the first throughbore having an axis corresponding to a first altered bone plane in the glenoid surface. A second throughbore opens into the contact surface, the second throughbore having an axis corresponding to a second altered bone plane in the glenoid surface. The axes of the first throughbore and of the second throughbore are not parallel to one another.

### CROSS-REFERENCE TO RELATED APPLICATION

The application is a continuation-in-part of U.S. patent application Ser. No. 17/088,713, filed on Nov. 4, 2020, which claims the priority of U.S. Patent Application No. 62/930,289, filed on Nov. 4, 2019, and incorporated herein by reference.

### TECHNICAL FIELD

The application relates computer-assisted surgery for the positioning of an augment in glenoid surgery.

### BACKGROUND

There are different challenges when performing shoulder arthroplasty in cases of severe glenoid deformity. Restoring the neutral glenoid alignment while preserving native bone may require the usage of bone grafts, augmented implants and/or wedges. The augmented implants intend to fill the void present on the pathologic side of the glenoid surface. Deformities are often present on the posterior or superior quadrant. Planning the implant position and orientation preoperatively allows the optimization of the location of the augment in order to reduce bone volume removal. The challenge is to reproduce the planned implant position and orientation intra-operatively to preserve native bone and/or to allow stable implantation of the glenoid component. There may consequently result a reduction of risks of complications in case of reverse shoulder surgery.

### SUMMARY

In one aspect, there is provided a computer-assisted surgery method for assisting a positioning of a baseplate in glenoid implant surgery comprising: obtaining a virtual model of a glenoid surface of a scapula; identifying a depth landmark in the glenoid surface of the scapula; obtaining a planned positioning of baseplate relative to the depth landmark; determining a bone alteration plan based on the planned positioning of the baseplate; and generating and outputting at least one patient-specific jig model representative of the bone alteration plan.

In another aspect, there is provided a computer-assisted surgery system for assisting a positioning of a baseplate in glenoid implant surgery comprising: a processing unit; and a non-transitory computer-readable memory communicatively coupled to the processing unit and comprising computer-readable program instructions executable by the processing unit for: obtaining a virtual model of a glenoid surface of a scapula, identifying a depth landmark in the glenoid surface of the scapula, obtaining a planned positioning of baseplate relative to the depth landmark, determining a bone alteration plan based on the planned positioning of the baseplate, and generating and outputting at least one patient-specific jig model representative of the bone alteration plan.

In yet another aspect, there is provided patient-specific instrumentation for reverse shoulder surgery comprising: a jig having a contact surface including a patient-specific surface portion negatively shaped as a function of a glenoid surface and configured to be applied against the glenoid surface in unique complementary engagement, a first throughbore opening into the contact surface, the first throughbore having an axis corresponding to a first altered bone plane in the glenoid surface, and a second throughbore opening into the contact surface, the second throughbore having an axis corresponding to a second altered bone plane in the glenoid surface, wherein the axes of the first throughbore and of the second throughbore are not parallel to one another.

### DESCRIPTION OF THE DRAWINGS

Reference is now made to the accompanying figures in which:

FIG. 1 is a perspective view of an augmented baseplate of a hemispherical ball joint in shoulder surgery;

FIG. 2 is a perspective view of an exemplary coordinate system for a glenoid of a scapula;

FIG. 3 is a flow chart of a computer-assisted surgery (CAS) method for assisting the positioning of an augment in glenoid implant surgery and for creating a PSI jig(s) in accordance with the present disclosure;

FIG. 4 is a screen shot of a graphic user interface used with a CAS method and system of the present disclosure showing a depth landmark;

FIG. 5 is another screen shot of a graphic user interface used with a CAS method and system of the present disclosure showing an implant relative to the depth landmark of FIG. 4;

FIG. 6 is a perspective view of a scapula with a first step of reaming with a first PSI jig;

FIGS. 7A and 7B are perspective views of a second PSI jig or PSI jig model incorporating a pair of non-parallel reaming axes;

FIG. 8 is a perspective view showing the second PSI jig or PSI jig model of FIGS. 7A and 7B relative to the scapula of FIG. 6;

FIG. 9 is a perspective view of another PSI jig that leverages inserted guide pins and implant drill guide to guide the location of an implant; and

FIG. 10 is a block diagram of a CAS system for assisting the positioning of an augment in glenoid implant surgery and for creating a PSI jig(s) in accordance with the present disclosure.

### DETAILED DESCRIPTION

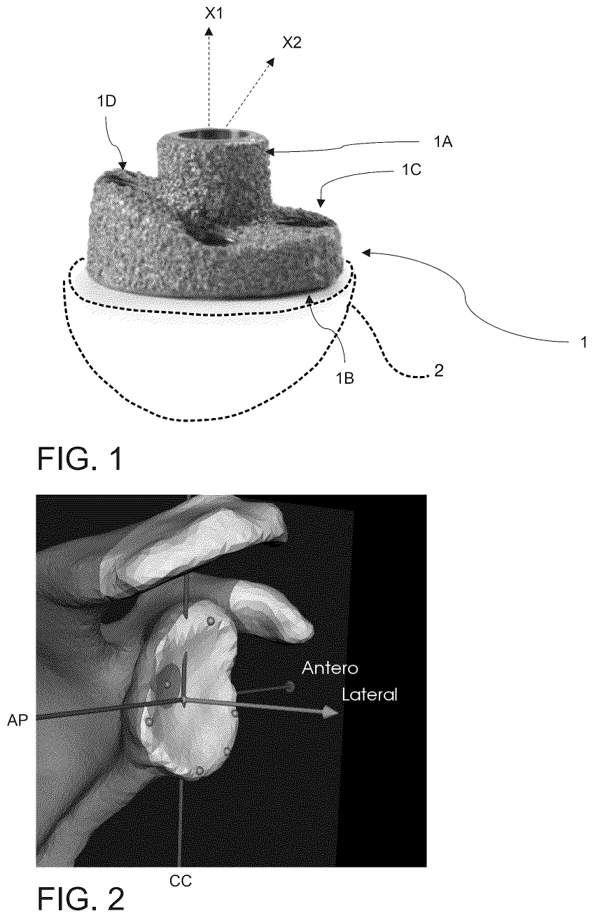

Referring to the drawings, and more particularly to FIG. 1, there is illustrated an augmented baseplate 1 that may be part of a glenoid implant, in reverse shoulder surgery, the glenoid implant including the augmented baseplate 1 and a hemispherical ball joint 2 fixed to the glenoid via the augmented baseplate 1—it may be male-female engagement for example. The augmented baseplate 1 is shown in a granular material, that may for instance be referred to as trabecular metal, but this is one option among others, as metal, graft, etc, may be used as well. In reverse shoulder surgery, the glenoid (a.k.a., glenoid vault, glenoid cavity, glenoid fossa, shown in FIG. 2) in the patient's scapula (a.k.a., shoulder blade) is implanted with a hemispherical ball joint, while the humerus defines the complementary spherical joint socket. Stated differently, reverse shoulder surgery may be defined in an aspect in a procedure in which a ball implant is secured to the glenoid, while a complementary plate implant is secured to the humerus, such that a center of rotation is tied to the glenoid as opposed to being in the humerus. For subsequent reference, a coordinate system is shown in FIG. 2 relative to the glenoid, with an anterior-posterior axis AP, a medio-lateral axis ML, and a cranial-caudal axis CC. In an aspect, axes AP and CC lie in the sagittal plane of the glenoid, with the ML axis normal to the sagittal plane. In another aspect, an origin of the coordinate system of FIG. 2 is located at a deepest point (most medial) of the glenoid, though this is optional.

The augmented baseplate 1 may thus be used as an interface between a resurfaced glenoid and the hemispherical ball joint 2, i.e., the hemispherical ball joint 2 is secured (a.k.a., fixed, implanted) to the glenoid, while a socket (not shown) is attached to the humerus. The augmented baseplate 1 may have a peg 1A that is received in a corresponding peg hole formed into the glenoid. More than one peg may be present and the augmented baseplate 1 may rely solely on fasteners as another possibility. The peg 1A may be tubular for a fastener (e.g., screw) to optionally secure the augmented baseplate 1 to the scapula, through the peg 1A. The body of the augmented baseplate 1 may be disc shaped, with an interface surface 1B for receiving the hemispherical ball joint thereon, with attachment holes distributed in the body of the augmented baseplate 1, for additional fasteners (e.g., screws) to optionally be used in securing the implant assembly to the scapula. The interface surface 1B may be circular in shape, and the body of the baseplate 1, from the interface surface 1B may be cylindrical or frusto-conical in shape, as examples. A central axis X1 of the baseplate 1, passing through the peg 1A, may be normal to the interface surface 1B in one aspect, though this may be otherwise. The bone interface surface of the body of the augmented baseplate 1 may have two surface portions, shown as 1C and 1D, with each surface portion 1C and 1D being generally planar. Stated differently, the surface portions 1C and 1D are two distinct planes that intersect, and that are in a non-parallel relation. The surface portions 1C and 1D are applied against the resurfaced glenoid when implanted, i.e., against a first altered bone plane and a second altered bone plane. In an aspect, surface portion 1C is parallel to the circular interface surface 1B, while the surface portion 1D is not. In another aspect, neither surface portions 1C and 1D are parallel to the circular interface surface 1B. The augmented baseplate 1 of FIG. 1 may be useful in reducing the amount of native bone that must be reamed off of the glenoid surface, notably by having the surface portion 1C lie against a minimally reamed portion of the glenoid. However, one of the challenges with the augmented baseplate 1 is that the glenoid must be reamed along two different axes to match the combined geometry of the surface portions 1C and 1D. One of the axes may be the central axis X1, and the other may be normal to the surface portion 1D when the augmented baseplate 1 is implanted, and is shown at X2.

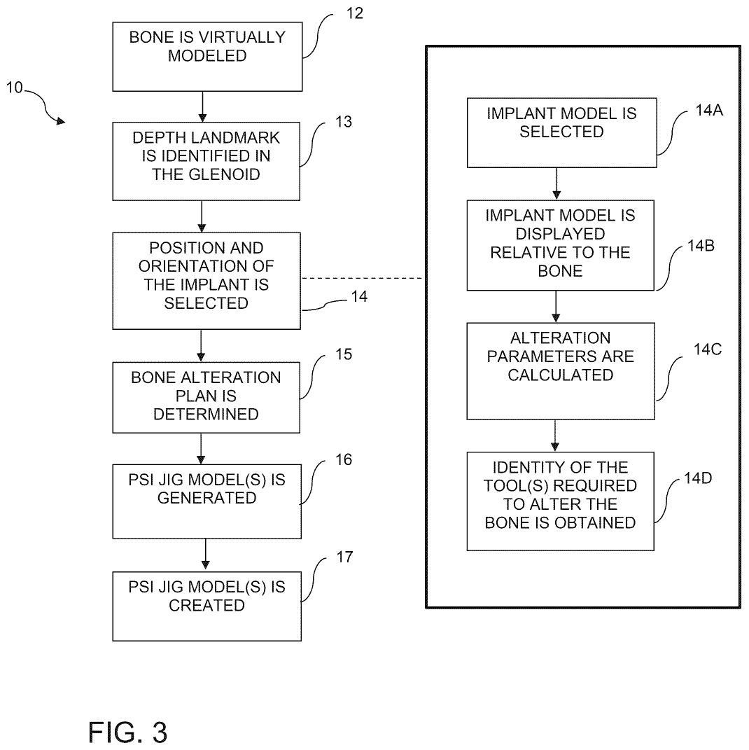

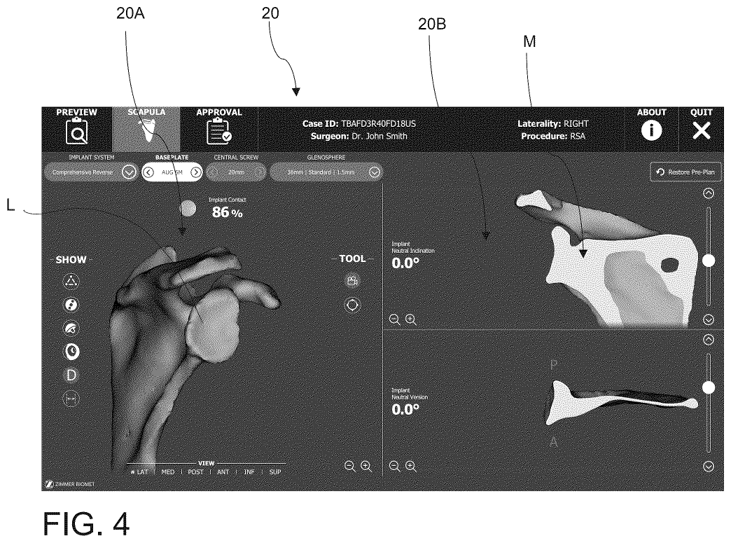

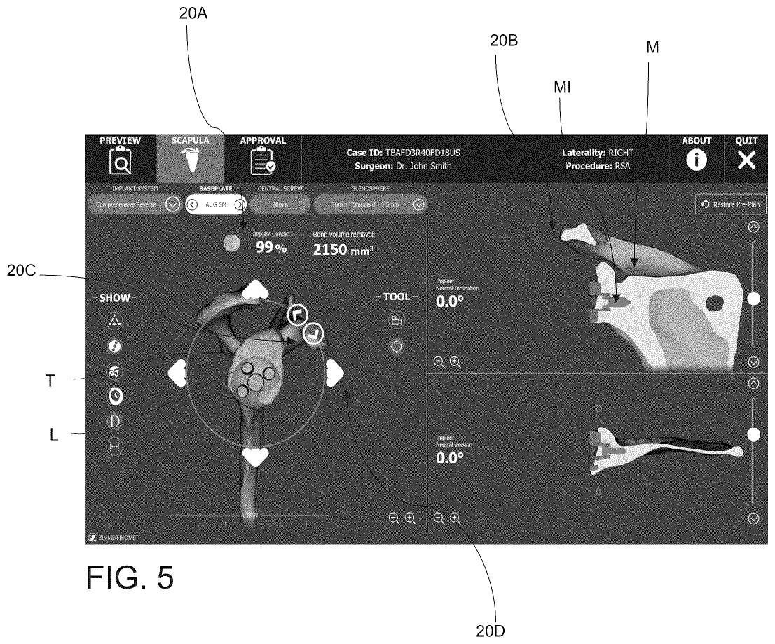

Referring to FIG. 3, a computer-assisted surgery (CAS) method for assisting the positioning of an augment or other implant component in glenoid implant surgery and/or for creating a patient specific jig(s) therefor, is generally shown at 10. The description that follows is applied to the augmented baseplate 1, but could also be applied to other implant components. The CAS method 10 may be performed entirely preoperatively, to plan an operation. Hence, reference to models herein may entail virtual 3D models, unless stated otherwise. For simplicity, reference will be made to the augmented baseplate 1, but the description extends to other implant components. The CAS method 10 may be driven by a CAS system described below in FIG. 9, which CAS system may provide an output in the form of a graphic user interface (GUI) 20 shown in FIGS. 4 and 5. The method 10 may for instance assist by planning a position and orientation of the augment and/or by creating a patient specific instrumentation (hereinafter PSI) jig for guiding an operator in altering the glenoid for subsequently anchoring the augment and implant to the glenoid. The method 10 may be used to assist with the positioning of the augmented baseplate 1 of FIG. 1 with its two non-parallel reaming axes. For clarity, reference to patient specific/PSI in the present application pertains to the creation of negative corresponding contour surfaces, i.e., a surface that is the negative opposite of a patient bone/cartilage surface, such that the patient specific surface conforms to the patient bone/cartilage surface, by complementary confirming unique engagement contact. The method is particularly suited to be used in shoulder surgery, when an implant must be secured to the glenoid cavity of the scapula (a.k.a., shoulder blade), with two-axis reaming.

According to 12, the bone is virtually modeled. This may include obtaining the model, which may also include generating the virtual model using imaging and may also include imaging the bone. The imaging may be done by any appropriate technology such as CT scanning (computerized tomography), fluoroscopy, or like radiography methods, 3D camera, providing suitable resolution of images. The bone modeling may also be performed or supplemented by surface palpation with a registration tool, as an alternative or supplemental aspect, using other tracking technology (e.g., optical, inertial sensors). The model of the bone comprises a surface geometry of parts of the bone with or without cartilage. As the present disclosure relates to thin bones, the modeling of the bone may comprise generating opposed surfaces to illustrate the depth profile of the portion of the bone of interest, i.e., the depth variations between the bone surfaces. The expression “depth” is used, as the bone will be altered in depth (e.g., using a drill); however, the expression “thickness” could also be employed, as in the thickness of the bone is profiled. The depth may be along the ML axis. The bone surfaces may include a proximal surface, that is exposed during surgery and upon which alterations are made, and a distal surface, often hidden behind soft tissue during surgery. To render surgery as minimally invasive as possible, the distal surface remains hidden so as not to displace soft tissue.

The bone modeling may comprise generating or refining a 3D surface of the bone if the bone modeling is not directly performed by the imaging equipment, or if not complete. Additional structures may be modeled as well, such as cartilage, etc. Referring to FIG. 4, the GUI 20 may display in a main panel 20A thereof the bone model M from different points of view (POVs), e.g., lateral, medial, posterior, anterior, inferior, superior, at the selection of an operator. FIG. 4 shows the bone model M from a lateral POV as an example in the main panel 20A. In an aspect, the bone model M is patient specific in the area of the glenoid. A remainder of the scapula may be absent or obtained from a bone atlas or from a generic model, as other features may not be as important.

According to 13, a depth landmark L is identified in the glenoid. The depth landmark L may for instance be a deeper point or surface in the baseplate footprint of the glenoid, i.e., the surface where it is anticipated that the baseplate 1 will be positioned. In an aspect, the depth landmark L is the deepest point or surface of the baseplate footprint. In yet another aspect, the depth landmark L may be in the form of a coordinate in the coordinate system of FIG. 2. Referring to FIG. 4, the depth landmark L is illustrated as a dot. The GUI 20 may provide sectional views in side panels 20B, to assist an operator in viewing bone slices from different POVs, for an operator to identify or view the depth landmark L. The side panels 20B may feature scroll scales to move along the slices. For example, the side panels 20B show a frontal plane cut (top side panel 20B), and a transverse plane cut (bottom side panel 20B), with the scroll scales allowing views to move along the AP axis and the CC axis, respectively. In an aspect, the depth landmark L is recorded and/or identified automatically by the CAS system, as a function of determined parameters, such as the deepest point of the glenoid.

As the method 10 may be used to minimize the amount of native bone to be reamed off, the identification of the depth landmark L as being one of the deeper points or surfaces of the glenoid, if not the deepest, may guide the subsequent planning in positioning the thicker parts of the augmented baseplate 1, i.e., where the surface portion 1D is, over the deeper or deepest points or surfaces of the glenoid. As a consequence, a minimized amount of bone may have to be reamed off. However, other approaches are contemplated, such as using the depth landmark L to indicate the “shallower” points or surfaces of the glenoid, i.e., the most lateral on the ML axis, to then position the thinner parts of the baseplate 1 over such shallower points.

According to 14 of FIG. 3, a position and orientation of the augmented baseplate 1 in the glenoid model M is selected. This may entail various substeps or steps, such as those shown in 14A-14D. Step 14 may include any one or any combination of the substeps 14A-14D.

According to 14A, based on the imaging, a baseplate model or other implant component may be selected using sizing parameters and like information, according to a surgeon's preference, to an engineer's design considerations, etc. The selection of the baseplate model may be based on baseplate stock geometries and on the depth profile of the glenoid, or like 3D geometry data obtained in 12. The size data for the augmented baseplate 1 may be obtained using a data file associated with the implant model or with the implant selection. The size data may also be calculated using the virtual implant model. The size data is specific to the implant selection or to the augmented baseplate selection. In an aspect, the selection of the baseplate model is executed automatically by the CAS system.

According to 14B, a model of the augmented baseplate 1 or other implant component may be displayed relative to the bone model M on the GUI 20, and to the depth landmark L. The display may be generated automatically by the CAS system. The CAS system may propose a position and orientation for the augmented baseplate 1, based on predetermined factors, such as minimum bone resurfacing, matching native joint position, restoring neutral glenoid alignment or native shoulder center of rotation, etc. The planned positioning (i.e., position and orientation) may also be selected by the operator, with the operator having the option of overriding the positioning set or proposed by the CAS system. To assist in the planning during the method 10, 14B may include generating a model of the augmented baseplate 1 relative to a virtual model M of the bone for navigated selection, i.e., allowing the operator and/or surgeon to move the implant or part of it relative to the bone, until a desired positioning is reached, i.e., the planned positioning. The planned positioning may include a position and orientation of the implant relative to the bone, whereby the navigated selection may include rotating and translating the virtual model of the implant relative to the virtual model of the bone. The rotating may be in one rotational degree of freedom relative to the ML axis (or also relative to the CC axis), while the translating may be in two translational degrees of freedom, in the sagittal plane (though movement in the frontal plane may also be considered). Referring to FIG. 5, the model of the augmented baseplate 1 is shown on the main panel 20A and the side panels 20B, relative to the bone model M. In the main panel 20A, navigation tools may be provided, for an operator to modify the position and/or orientation, relative to the depth landmark L. In an aspect, the model of the augmented baseplate 1 has a marker T indicative of the thicker point of the augmented baseplate 1 (i.e., at the periphery of the surface portion 1D), hence the marker T may be referred to as an orientation marker for the augmented baseplate 1—the marker T being the arrowhead or other symbol. Alternatively or additionally, the marker T could show the thinner part of the augmented baseplate 1, etc. Orientation adjustments, e.g., a rotation of the augmented baseplate 1 relative to the ML axis may be done via scroll wheel 20C. It may also be possible to tilt the sagittal plane, based on CAS system guidance or on operator input. The tilting of the sagittal plane may provide two additional rotational degrees of freedom of adjustment. Position adjustments, e.g., a location of the augmented baseplate 1 in the sagittal plane or any other plane of reference, may be done via scroll arrows 20D.

As an optional part of 14B, in the side panels 20B, a depth of the augmented baseplate 1 relative to the bone model M may be displayed. This may include a visual representation of the cement bore and of a central fixation screw, in the form of model M1. The central fixation screw model M1 consists of a representation of the screw axis that must be drilled in the glenoid, for the augmented baseplate 1 and other implant components, such as the fasteners, to be received and anchored to the bone based on a planned positioning of the augmented baseplate 1. As another possibility, a cement bore model may be displayed, with or without the screw model M1, and may comprises a bore or mantel in which the cement will be received. It is observed that a depth of the central fixation screw M1 exceeds the depth of the peg 1A of the augmented baseplate 1, and may also exceed the sectional size of the implant components. Hence the illustration of the central fixation screw model M1 and/or of the cement bore may be useful to ensure that the bone is not pierced through or to confirm exit point on the scapula.

According to 14C, alteration parameters are calculated for the current position and orientation of the augmented baseplate 1 relative to the bone model M. The alteration parameters may include the volume of bone removal based on the current position and orientation. The volume of bone removal may be automatically calculated by virtually overlaying the model of the augmented baseplate 1 over the bone model M in the current position and orientation, i.e., that shown on the GUI 20. The overlaying results in an overlapping volume indicative of the bone matter that must be removed. Stated differently, the volume of bone removal corresponds to the subtraction between the native scapula volume and the scapula volume after reaming. If the position and orientation of the augmented baseplate 1 is modified as per 14B, the volume of bone removal may be adjusted in real-time. In an aspect, the CAS system indicates the volume of bone removal as a function of the least possible volume of bone removal, for instance as a percentage. The CAS system may also automatically set a position and orientation of the augmented baseplate 1 based on the least possible volume of bone removal, with a possibility for an operator to override the automatic setting. In 14C, another parameter may be the contact surface of the augmented baseplate 1 with the bone, taking into consideration the reaming that would be performed. Indeed, because of some surface deformities, pathologies or abnormalities, some parts of the native bone may be medially inward of reaming planes, and hence result in an absence of contact. Accordingly, the contact surface may be a percentage value indicative of how much of the surface portions 1C and 1D contact the bone at the current virtual position and orientation of the augmented baseplate 1, taking into consideration the planned reaming. Other alteration parameters may be calculated, such as the deviation from the neutral glenoid alignment. The augment size and positioning may thus be refined using the alteration parameters calculated in 14C.

According to 14D, with the position and orientation of the augmented baseplate 1 selected, an identity of the tool(s) required to alter the bone may be obtained, optionally. The CAS system may automatically determine the identity of the tool(s), based on the planned positioning of the selected implant, and the determination may be based on the size data of the selected implant. For example, if a peg of a given diameter and length is to be inserted in the bone, the identity of the reaming tool will be as a function of making a hole of sufficient cross-section to receive the peg. The pairing of implants and altering tool(s) may be done before the 14C, for example as part of the specifications of the implants. The specifications may indeed identify the tool(s) required or suggested to perform the alterations and prepare the bone to receive the selected implant. The identity may be part of a data file accompanying the implant model obtained by the CAS system. The determination of identity may also be effected once the implant is selected, based on a condition or anatomical features of the bone.

According to 15, a bone alteration plan is determined, as a function of the position and orientation of the implant selected in 14, and of the bone alterations required to achieve the selected position and orientation. In the aspect of the augmented baseplate 1, the bone alteration plan may include a position and orientation (trajectory) of both reaming and/or drilling axes to perform the two-step reaming described above. The bone alteration plan may include identifying reamer dimensions or type to be paired to the selected implant. However, the reamer dimensions and type may have been selected in 14D as well. The geometry data of reamers may be that of the working end of the tool(s), i.e., the part of the tool(s) that alter the bone. The geometry data may be in the form of a virtual tool model and/or quantitative data. The bone alternation plan of FIG. 15 may include the positioning and orienting of guide landmarks, such as guide pins that will define the trajectory of the drill and/or reaming tools. In an aspect, one or more of the guide pins are guidewires for cannulated reamers.

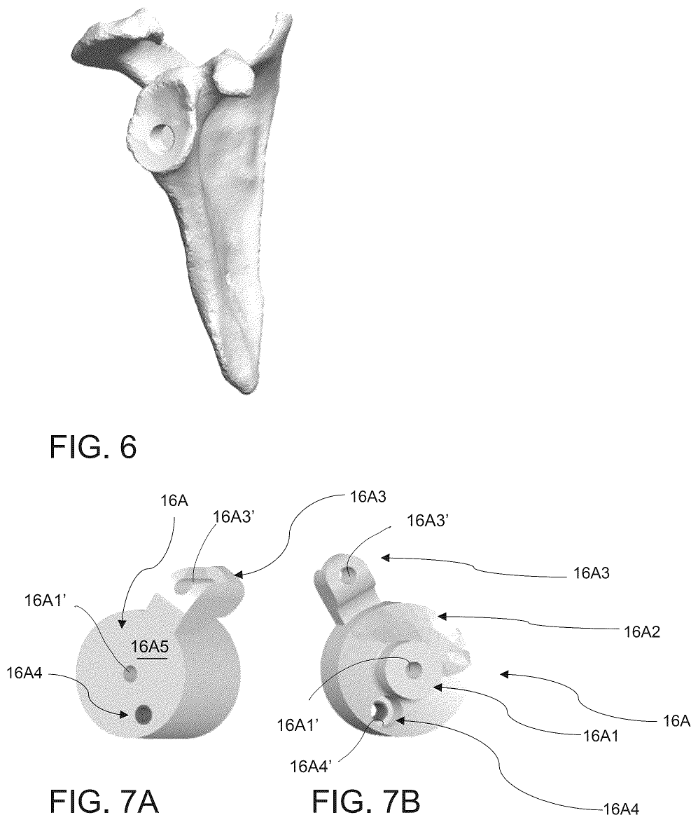

In 15, a depth image or model may be output, displaying the image or model of the virtual model of the bone as altered, for instance in various steps of alteration. The side panel images of FIG. 5, with or without the augmented baseplate 1 may provide a 2D view of the bone in depth. FIG. 6 shows an exemplary model of the bone, after a first of two reaming steps. The 2D views may be extracted from 3D models, to show the penetration of the implant, e.g., the augmented baseplate 1, relative to the depth of the bone. The virtual model of the bone may be used to create a physical model of the bone as altered. The physical model of the bone as altered may be used by an operator with implants to mechanically test the fit of the baseplate 1 on the physical model, to determine if the fit is appropriate. The physical model of the bone may be fabricated with appropriate techniques, such as 3D printing, CNC machining, etc.

According to 16, a PSI jig model(s) may be generated for the selected position and orientation of the implant, such as the augmented baseplate 1. The jig model will have a contact surface(s) defined to abut against the bone based on the planning of 14 and 15. Typically, the PSI jig may include a drilling guide or landmark placement guide that will assist the identified tool(s) of 13 to alter the bone to ensure the implant is positioned and oriented as planned, i.e., to ensure that the alterations are as planned. The PSI jig model of 16 may therefore comprise cutting planes, drill guides, slots, or any other tooling interface or tool, oriented and/or positioned to allow bone alterations to be made in a desired location of the bone, relative to the preplanned position. Moreover, as the depth of the reaming planes must be as planned, the PSI jig model of 16 may feature a depth stop for the tool, or like tool abutment surfaces to limit the depth of machining of the tool as a function of the planned cement bore depth. The PSI jig model of 16 may be a 3D printable model (e.g., an STL file). Examples of PSI jig models that may be created in 16 and fabricated in 17 may be as in United States Patent Application No. 2015/0073424, incorporated herein by reference. As another example, a first PSI jig model that is created in 16 and fabricated in 17 is as shown at 416 in U.S. Pat. No. 9,615,840, incorporated herein by reference. The PSI jig model 416 thereof may be used to place a pair of guide pins in the scapula, with a first guide pin P1 being centered at an eventual location of the peg hole, and a second guide pin P2 being in a secondary zone that is out of the implant footprint. The first guide pin P1 may be used to define the peg hole in the glenoid, using for example a cannulated reamer or drill bit slid over the first pin. As part of the machining of the peg hole, or after such step, the first guide pin P1 may then be used to ream a first of the two surfaces of the resurfaced glenoid. This first of the two surfaces may be the one on which the surface portion 1C of the augmented baseplate 1 will lie. Consequently, if the PSI jig model 416 of U.S. Pat. No. 9,615,840 were used, with subsequent reaming of the first surface, the glenoid could reach the state of FIG. 6. The pins are removed for clarity but may be present when the glenoid is in the state of FIG. 6, intraoperatively (i.e., after the method 10 has been completed).

The guide pins may define a trajectory for PSI jigs that will be subsequently used, such as the one shown in FIGS. 7A and 7B. FIGS. 7A and 7B illustrate a second PSI jig 16A, that may be generated as a model in 16, and fabricated as per 17 described below. The PSI jig 16A could be used alone as well, i.e., without any other PSI jig. The second PSI jig 16A has a body optionally with a peg portion 16A1 surrounded by and projecting from a PSI surface 16A2, i.e., negatively shaped as a function of the bone surface it will contact. The peg portion 16A1 may also be said to have a patient specific geometry as it is shaped to be complementarily received in the peg hole in the glenoid, as in FIG. 6, if present. The peg portion 16A1 may have a throughbore 16A1′ for being slid onto a first guide pin P1 indicative of a center of the peg hole in the bone. The throughbore 16A1′ may open into the PSI surface 16A2 if no peg portion is present. As observed, the PSI surface 16A2 may have a relatively planar portion that will rest in unique complementary contact with the reamed first surface of the glenoid of FIG. 6, while another portion of the

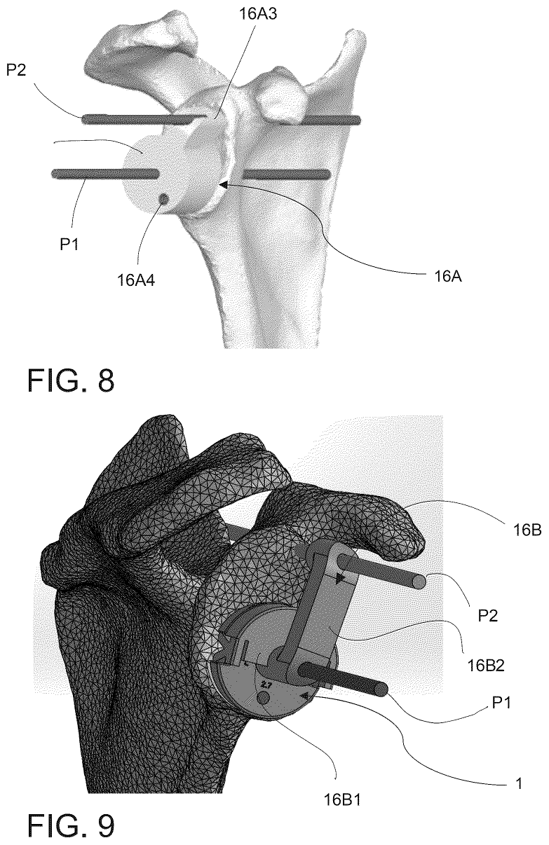

PSI surface 16A2 is irregular, i.e., it conforms to the non-machined or non-altered part of the glenoid. Therefore, when the PSI jig 16A is positioned against the reamed glenoid of FIG. 6, a unique complementary engagement is reached, as in FIG. 8, i.e., only one engagement is possible. A tab 16A3 or like portion having a throughbore 16A3′ may be slid onto the secondary guide pin P2, with the secondary guide pin P2 being optional and assisting in achieving the proper orientation of the PSI jig 16A on the bone. The tab 16A3 may project out of a planned footprint of the augmented baseplate. The PSI jig 16A may then be used for its guide throughbore 16A4. The guide throughbore 16A4 may be used to place another pin in the scapula, which pin has a trajectory aligned with the second reaming axis (e.g., parallel). The second reamingi axis is used to ream the surface of the glenoid against which will lie the surface portion 1D of the augmented baseplate 1. A cannulated reamer may be used, with an appropriate stopper, for this purpose, as guided by a pin providing the trajectory of the second reaming axis. Alternatively, the guide hole 16A4 may be used to drill a pilot hole in the glenoid, which pilot hole may be indicative of a location of the second reaming axis. Accordingly, it is contemplated to provide a wear sleeve (e.g., metal) if the body of the PSI jig 16A is made of a polymer. Moreover, a counterbore, as shown in FIG. 7B, or like receiving volume, may be present at the opening of the guide hole 16A4 in the PSI surface 16A2, for bone debris to accumulate during the drilling, and/or to facilitate the insertion of the sleeve therein. The drilling of a pilot hole may be desired if the pins P1 and P2 are kept on the scapula, to enable the removal of the PSI jig 16A. The pins P1 and P2 positioned with the first PSI jig model could be used in the manner shown in FIG. 9, to place or impact the augmented baseplate 1 against the resurfaced glenoid, with a positioning jig 16B to which the baseplate 1 is clipped. Therefore, the PSI jig 16A has at least bores respectively representing the reaming axes, with the bores having their central axes non-parallel to one another, to emulate the X1 and X2 arrangement of the augmented baseplate 1. The PSI jig 16A has a given thickness between the PSI surface 16A2 and an opposite top surface 16A5, such that pins or drills received in the bores will have a trajectory generally corresponding to a central axis of the bores. In an embodiment, the PSI jig 16A has a periphery between the surfaces 16A2 and 16A5 that gives the PSI jig 16A a footprint equivalent to that of the base plate.

Referring to FIG. 9, the positioning jig 16B is shown having a clip portion 16B1 that releasably receives the baseplate 1. The clip portion 16B1 may configured to connect in a complementary manner with the baseplate 1, for the baseplate 1 to be in a known orientation when connected to the clip portion 16B1. For example, the clip portion 16B1 may cooperate with screw bores in the baseplate 1. The clip portion 16B1 is one possible configuration that may be employed to releasably connect the baseplate 1 to the positioning jig 16B, with other configurations including fingers, a cup, a collar, etc. The positioning jig 16B may be said to be patient specific, in that an arm 16B2 extending between pins P1 and P2, and therefore using the trajectory of these pins, may be fabricated based on patient specific planning. The arm 16B2 has holes to receive therein the pins P1 and P2. Therefore, when the baseplate 1 is connected to the clip portion 16B1, the arm 16B2 may be slid onto the pins P1 and P2, for the baseplate 1 to come into contact with the resurface glenoid in the planned manner. As observed, a screw hole of the baseplate 1 is exposed when the baseplate 1 is clipped to the positioning jig 16B, for a screw to be used to secure the baseplate 1 to the resurface glenoid. The positioning jig 16B may then be removed.

As an alternative or in addition to the creation of PSI jig models, in 16, a navigation file may be created, which navigation file will be used during surgery to guide the operator or robot in manipulating the tools to alter the bone as planned in 14. For example, inertial sensors or optical tracking technology may be used in the implant procedure, and the navigation file will be used by the computer-assisted surgery system to guide the operator to alter the bone in a manner corresponding to the planning of 14.

According to 17, once the PSI jig model(s) has been generated, the PSI jig(s) may be created, according to any appropriate method, such as 3D printing (additive manufacturing), NC machining, etc. The PSI jig created in 17 may then be used intra-operatively to allow alterations to be made on the bone, and to reproduce the planned reaming planes. 17 may include driving an apparatus to fabricate the PSI jig(s). For example, to ensure a suitable depth is achieved, the PSI jig may be used to guide a drill (e.g., a cannulated drill) or a pressurizer. The PSI jig(s) may consequently be used in the manner shown in FIGS. 6 to 9, based on the planning done in the method 10 of FIG. 3.

Now that the method for assisting the positioning of an augment in glenoid implant surgery for creating a PSI jig(s), a system is set forth.

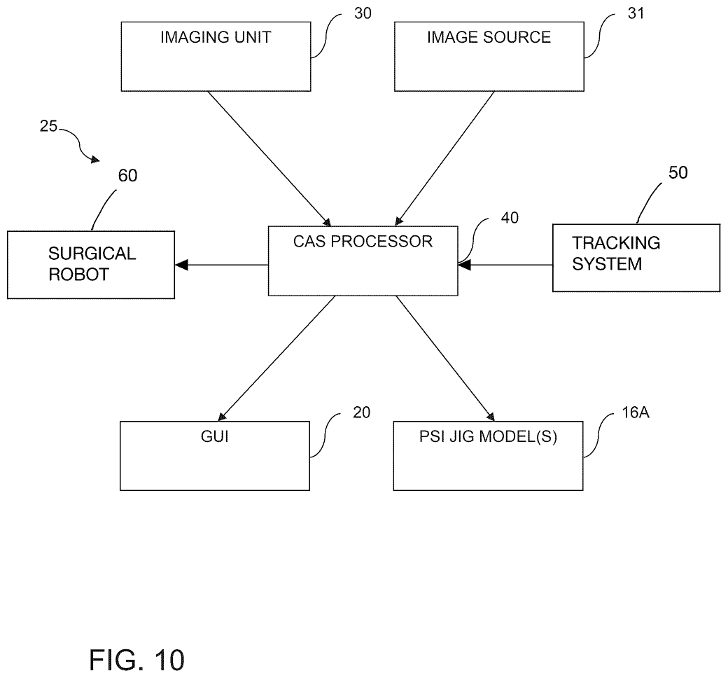

Referring to FIG. 10, a system for assisting the positioning of an augment in glenoid implant surgery and for creating a PSI jig(s) therefor, is generally shown at 25 in FIG. 10. The system 25 may include the GUI 20 described above, and may also include the various modules to generate at least some of the data shown in FIGS. 4 and 5. The system 25 may have an imaging unit 30, such as a CT scan or an X-ray machine (2D or 3D), MRI, so as to obtain images of the bone and implant. As an alternative, images may be obtained from an image source 31. As an example, a CT scan or other imaging modality may be operated remotely from the system 25, whereby the system 25 may simply obtain images and/or processed bone and implant models from the image source 31. The images may also include images from other sources, including surface palpation data obtained from tracking technology that may be part of the imaging unit 30 and/or may contribute in creating the images of the image source 31. The imaging unit 30 has the capacity of modeling a 3D model of the bone including opposed surfaces to illustrate the depth profile of the portion of the bone of interest.

The system 25 comprises a processor unit 40 (e.g., computer, laptop, etc.) that comprises different modules so as to ultimately produce a jig model, fabrication file therefor, or a navigation file. The processing unit 40 of the system 25 may therefore a non-transitory computer-readable memory communicatively coupled to the processing unit 40 and comprising computer-readable program instructions executable by the processing unit 40 for performing at least some of the steps of the method 10 of FIG. 3. Consequently, the processing unit 40 may output its data via the GUI 20 which may take any appropriate form (e.g., monitor, screen, tablet, smart device, etc). The system 25 may also output a PSI jig model or navigation file that will be used to create the PSI jig. The PSI jig may be created, according to any appropriate method, such as 3D printing (additive manufacturing), NC machining, etc. The PSI jig or navigation file is then used intra-operatively to alter the bone for subsequent implant installation. A tracking system 50 may track tools and bones during the surgical procedure. The tracking system 50 may be an optical tracking system featuring trackers on the bones and tools and a tracking camera, such as the Navitracker® system. Other tracking modalities are possible, including standard cameras, depth cameras (e.g., Kinect®), QR codes, etc. The processing unit 40 may drive the surgical robot 60 to perform the alterations.

Thus, based on the above description, the processing unit 40, optionally using the tracking system 50 and/or the robot 60 may be used in computer assistance to perform a method for positioning a glenoid implant with computer assistance that may include: obtaining a virtual model of a glenoid surface of a scapula, identifying a depth landmark in the glenoid surface of the scapula, obtaining a planned positioning of baseplate relative to the depth landmark, determining a bone alteration plan based on the planned positioning of the baseplate; reaming the glenoid surface of the scapula based on the bone alteration plan; securing a baseplate to the reamed glenoid surface according to the planned positioning; and securing a ball joint to the baseplate. This may include: including generating and outputting at least one patient-specific jig model representative of the bone alteration plan; receiving data a volume of bone removal as a function of the planned positioning; reaming the glenoid surface of the scapula based on the bone alteration plan includes reaming along two non-parallel reaming axes to define two reaming planes in the glenoid; generating and outputting the at least one patient-specific jig model representative of the bone alteration plan includes generating one said patient-specific jig model with guides for the two non-parallel reaming axes; obtaining a physical patient-specific jig corresponding to said patient-specific jig model and positioning said patient-specific jig with the guides for the two non-parallel reaming axes; reaming the glenoid surface of the scapula based on the bone alteration plan includes reaming along the two non-parallel reaming axes to define two reaming planes in the glenoid; reaming the glenoid surface of the scapula based on the bone alteration plan includes using a robot arm positioned relative to the scapula as a function of the bone alteration plan; identifying a depth landmark in the glenoid surface of the scapula includes receiving an indication of a deepest point in the glenoid surface; driving an apparatus for fabricating at least one patient-specific jig from the at least one patient-specific jig model; driving the apparatus for fabricating the at least one patient-specific jig includes fabricating the at least one patient-specific jig having a contact surface including a patient-specific surface portion negatively shaped as a function of a glenoid surface and configured to be applied against the glenoid surface in unique complementary engagement, a first throughbore opening into the contact surface, the first throughbore having an axis corresponding to a first altered bone plane in the glenoid surface, and a second throughbore opening into the contact surface, the second throughbore having an axis corresponding to a second altered bone plane in the glenoid surface, wherein the axes of the first throughbore and of the second throughbore are not parallel to one another; fabricating the at least one patient-specific jig includes fabricating the at least one patient-specific jig such that the contact surface includes a peg portion, the first throughbore opening into the peg portion; fabricating the at least one patient-specific jig includes fabricating the at least one patient-specific jig such the contact surface includes a planar portion; fabricating the at least one patient-specific jig includes fabricating the at least one patient-specific jig such that a third throughbore is present, the third throughbore defining an axis parallel to the axis of the first throughbore; securing the baseplate to the reamed glenoid surface includes securing an augmented baseplate having an implant interface surface, and a bone interface surface, the bone interface surface having two planes in a non-parallel relation respectively corresponding to the first bone plane and the second bone plane; and/or positioning guide pins using the patient-specific jig with the guides and using the guide pins for reaming along the two non-parallel reaming axes to define two reaming planes in the glenoid.

While the methods and systems described above have been described and shown with reference to particular steps performed in a particular order, these steps may be combined, subdivided or reordered to form an equivalent method without departing from the teachings of the present disclosure. Accordingly, the order and grouping of the steps is not a limitation of the present disclosure.

1. A method for positioning a glenoid implant with computer assistance comprising:
obtaining a virtual model of a glenoid surface of a scapula, identifying a depth landmark in the glenoid surface of the scapula, obtaining a planned positioning of baseplate relative to the depth landmark, determining a bone alteration plan based on the planned positioning of the baseplate;
reaming the glenoid surface of the scapula based on the bone alteration plan;
securing a baseplate to the reamed glenoid surface according to the planned positioning; and
securing a ball joint to the baseplate.

2. The method according to claim 1, further including generating and outputting at least one patient-specific jig model representative of the bone alteration plan.

3. The method according to claim 1, further comprising receiving data a volume of bone removal as a function of the planned positioning.

4. The method according to claim 1, wherein reaming the glenoid surface of the scapula based on the bone alteration plan includes reaming along two non-parallel reaming axes to define two reaming planes in the glenoid.

5. The method according to claim 2, wherein generating and outputting the at least one patient-specific jig model representative of the bone alteration plan includes generating one said patient-specific jig model with guides for the two non-parallel reaming axes.

6. The method according to claim 5, further including obtaining a physical patient-specific jig corresponding to said patient-specific jig model and positioning said patient-specific jig with the guides for the two non-parallel reaming axes.

7. The method according to claim 6, wherein reaming the glenoid surface of the scapula based on the bone alteration plan includes reaming along the two non-parallel reaming axes to define two reaming planes in the glenoid.

8. The method according to claim 1, wherein reaming the glenoid surface of the scapula based on the bone alteration plan includes using a robot arm positioned relative to the scapula as a function of the bone alteration plan.

9. The method according to claim 1, wherein identifying a depth landmark in the glenoid surface of the scapula includes receiving an indication of a deepest point in the glenoid surface.

10. The method according to claim 2, further comprising driving an apparatus for fabricating at least one patient-specific jig from the at least one patient-specific jig model.

11. The method according to claim 10, wherein driving the apparatus for fabricating the at least one patient-specific jig includes fabricating the at least one patient-specific jig having a contact surface including a patient-specific surface portion negatively shaped as a function of a glenoid surface and configured to be applied against the glenoid surface in unique complementary engagement, a first throughbore opening into the contact surface, the first throughbore having an axis corresponding to a first altered bone plane in the glenoid surface, and a second throughbore opening into the contact surface, the second throughbore having an axis corresponding to a second altered bone plane in the glenoid surface, wherein the axes of the first throughbore and of the second throughbore are not parallel to one another.

12. The method according to claim 11, wherein fabricating the at least one patient-specific jig includes fabricating the at least one patient-specific jig such that the contact surface includes a peg portion, the first throughbore opening into the peg portion.

13. The method according to claim 11, wherein fabricating the at least one patient-specific jig includes fabricating the at least one patient-specific jig such the contact surface includes a planar portion.

14. The method according to claim 11, fabricating the at least one patient-specific jig includes fabricating the at least one patient-specific jig such that a third throughbore is present, the third throughbore defining an axis parallel to the axis of the first throughbore.

15. The method according to claim 1, wherein securing the baseplate to the reamed glenoid surface includes securing an augmented baseplate having an implant interface surface, and a bone interface surface, the bone interface surface having two planes in a non-parallel relation respectively corresponding to the first bone plane and the second bone plane.

16. The method according to claim 7, including positioning guide pins using the patient-specific jig with the guides and using the guide pins for reaming along the two non-parallel reaming axes to define two reaming planes in the glenoid.
